(19) United States
(12) Patent Application Publication
(10) Pub. No.: US 2023/0320798 A1
(43) Pub. Date: Oct. 12, 2023

(54) JOINT CONTROL IN A MECHANICAL SYSTEM

(71) Applicant: CMR SURGICAL LIMITED, Cambridge, Cambridgeshire (GB)

(72) Inventors: Martin Joseph Blake, Cambridge (GB); Andrew John Hazell, Cambridge (GB)

(21) Appl. No.: 18/012,771

(22) PCT Filed: Jul. 5, 2021
(86) PCT No.: PCT/GB2021/051699

§ 371 Date: Dec. 23, 2022

(30) Foreign Application Priority Data
Jul. 6, 2020 (GB) 2010355.2

(51) Int. Cl. A61B 34/37; B25J 9/16

(52) U.S. Cl. CPC A61B 34/37; B25J 9/1633; A61B 2090/066

(57) ABSTRACT

A controller for controlling the configuration of a joint in a surgical robot, the joint being driven by a drivetrain which transfers power from a drive source to the joint, wherein the controller is configured to: receive a first input indicating a configuration of the drive source; receive a second input from a first sensor, the second input indicating a measured configuration of the joint in the surgical robot; calculate a value of output torque about the joint using the first input and the second input; and calculate, using the value of output torque, a value of input torque to be applied to the joint in the surgical robot by the drive source.

### FIELD OF THE INVENTION

This invention relates to controlling the configuration of the joint in a mechanical system such as a robot arm, and in particular to a controller that controls this configuration without using torque sensor data as an input.

### BACKGROUND OF THE INVENTION

A typical robotic manipulator comprises a series of rigid elements that are coupled together by one or more joints, which may be joined in series to form an arm. The manipulator further comprises a base unit at a first end of the series of rigid elements, and an end effector at a second end that opposes the first end. Each joint of the manipulator is driven by a drive source and a corresponding drivetrain so as to cause relative motion of the rigid elements. This relative motion is used to vary the configuration of the end effector at a desired location. Each joint may provide rotational motion and/or linear motion. The drive source may be any suitable means, such an electronic motor or a hydraulic actuator.

In theoretical modelling, the drivetrain that actuates a robotic joint can be modelled as “stiff”. A stiff drivetrain is one that does not experience any stiction or backlash. Backlash refers to the lost motion that is experienced when due to a change in direction and is caused by gaps or clearance between interfacing components within the drivetrain. If there is no stiction or backlash in the drivetrain, then the torque generated by the drive source (i.e. the input torque that is provided to a first end of the drivetrain) will always be proportional to the torque that is experienced by the joint (i.e. output torque at a second end of the drivetrain that opposes the first end).

In reality, the components of the drivetrain that actuate a robotic joint have an associated level of elasticity. Thus, the stiffness of the joint cannot be accurately modelled using a linear relationship between input torque and output torque. It is therefore necessary to control the drive source using an algorithm that incorporates multiple feedback terms, those feedback terms corresponding to sensor data measured at both the input and output of the drivetrain. Sensor data can therefore be used to provide an indication of the performance of the joint. The incorporation of multiple feedback terms into a control algorithm increases the complexity of a controller associated with that algorithm and also introduces stability limitations.

There is a need fora simpler method of controlling the configuration of a joint in a mechanical system, such as a robot arm.

### SUMMARY OF THE INVENTION

According to a first aspect, there is provided a controller for controlling the configuration of a joint in a surgical robot, the joint being driven by a drivetrain which transfers power from a drive source to the joint, wherein the controller is configured to: receive a first input indicating a configuration of the drive source; receive a second input from a first sensor, the second input indicating a measured configuration of the joint in the surgical robot; calculate a value of output torque about the joint using the first input and the second input; and calculate, using the value of output torque, a value of input torque to be applied to the joint in the surgical robot by the drive source.

The first input may be received from a second sensor and may indicate a measured configuration of the drive source.

The measured configuration of the drive source may be the measured physical position of the drive source, and the measured configuration of the joint may be the measured physical position of the joint.

The controller may be further configured to receive a third input indicating a desired configuration for the joint.

The desired configuration may be the desired physical position of the joint.

The value of input torque may be calculated using a comparison between the first input and the third input.

The value of input torque may be calculated using a comparison between the second input and the third input.

The value of input torque may be calculated using a comparison between a first derivative of the first input and a first derivative of the third input.

The value of input torque may be calculated using a comparison between a first derivative of the second input and a first derivative of third input.

The output torque may be characterised by a relationship between an elongation value of the drive train and a stiffness value of the drivetrain.

The value of output torque may be represented by the equation τₒ=kφ(qᵢ−qₒ), where qᵢ is the first input, qₒ is the second input and kφ is a spring coefficient of the drivetrain.

kφ may be related to (qᵢ−qₒ) by a continuous function.

kφ may be selected from a discrete range of values, each value of kφ being associated with a range of elongation values that are defined by one or more predetermined threshold values.

The spring coefficient may be selected from three distinct values in dependence on a measured elongation of the drivetrain, wherein: a first value is selected for the spring coefficient if the value of measured elongation is below a first predetermined threshold; a second value is selected for the spring coefficient if the value of measured elongation is above the first predetermined threshold and below a second predetermined threshold; and a third value is selected for the spring coefficient if the value of measured elongation is above the second predetermined threshold.

The range of elongation values that is above the first predetermined threshold and below a second predetermined threshold may correspond to a backlash region of the joint.

The value of input torque may be represented by the following equation:

τᵢ=kₚₒ(qᵣqₒ)+kₚᵢ(qᵣ−qᵢ)+k_{do}(\dot{q}ᵣ−\dot{q}ₒ)+k_{di}(\dot{q}ᵣ−\dot{q}ᵢ)−kₜkφ(qᵢ−qₒ)

Wherein qᵢ is the first input, qₒ is the second input, qᵣ is the third input and kₚₒ, kₚᵢ, k_{do}, k_{di} and kₜ are gains associated with the first, second and third inputs.

The controller may be configured to repeatedly calculate the value of output torque.

The controller may be implemented within a dynamic torque observer, the dynamic torque observer being configured to calculate a value of dynamic torque by applying a weighting to the value of output torque calculated by the controller.

Calculating the value of input torque may comprise subtracting a torque term which compensates for the action of gravity from the value of output torque.

The drive source may be a motor.

The first sensor may be located at, or at a position proximal to, a second end of the drive train at which the joint is located.

The second sensor may be located at, or at a position proximal to, a first end of the drive train at which the drive source is located.

The drivetrain may comprise one or more gears.

The drivetrain may be a harmonic drive.

According to a second aspect, there is provided a method (e.g. a computer-implemented method) for controlling the configuration of a joint in a surgical robot, the joint being driven by a drivetrain which transfers power from a drive source to the joint, the method comprising: receiving a first input indicating a configuration of the drive source; receiving a second input from a first sensor, the second input indicating a measured configuration of the joint in the surgical robot; calculating a value of output torque for the joint using the first input and the second input; and calculating, using the value of output torque, a value of input torque to be applied to the joint in the surgical robot by the drive source.

### DETAILED DESCRIPTION

The present invention will now be described by way of example with reference to the accompanying drawings. In the drawings:

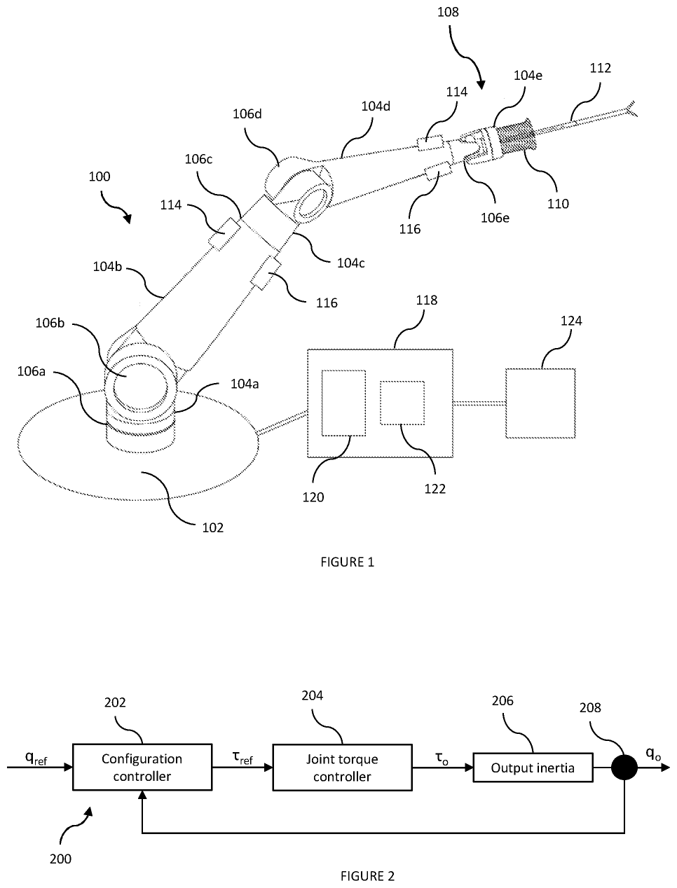

FIG. 1 illustrates the configuration of a robot arm;

FIG. 2 is a block diagram illustrating an idealised control loop for controlling the configuration of a joint in a robot arm;

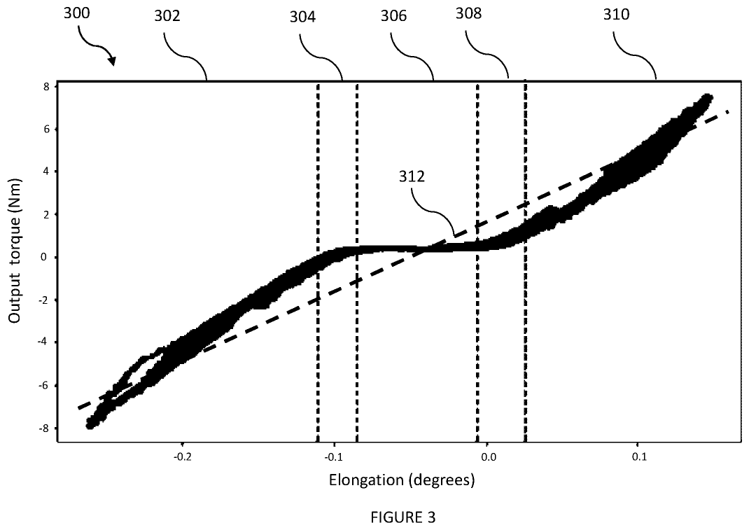

FIG. 3 illustrates the characterisation of the non-linear stiffness of a drivetrain for a robotic joint;

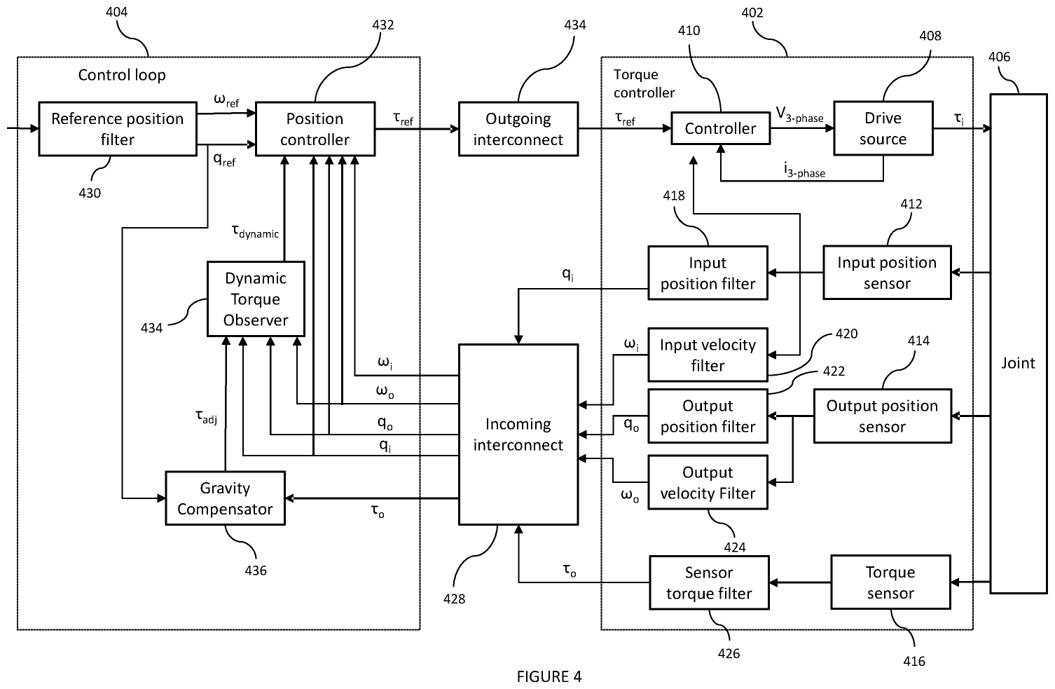

FIG. 4 is a block diagram illustrating a full state control scheme for controlling the configuration of a joint in a robot arm;

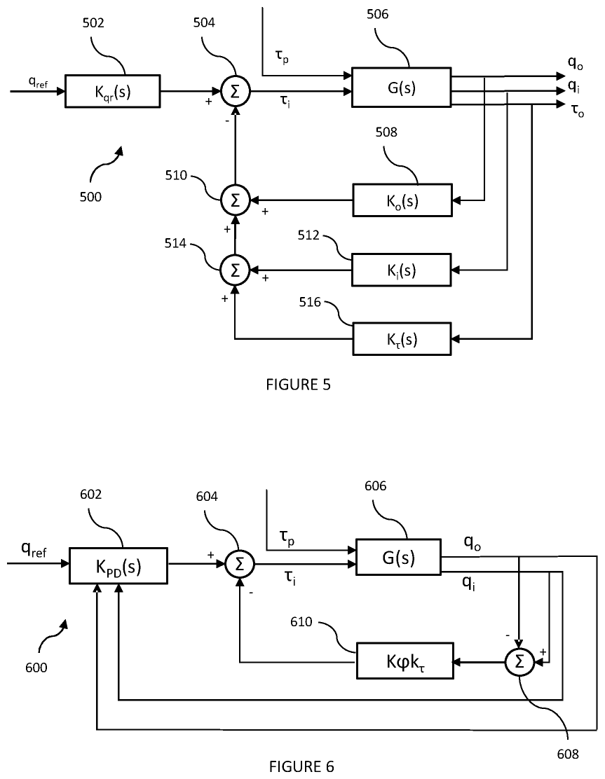

FIG. 5 is a block diagram illustrating a control algorithm for a controller as illustrated in FIG. 4, the control algorithm using torque sensor data as an input;

FIG. 6 is a block diagram illustrating a control algorithm for controlling the configuration of a joint in a robot arm that does not use torque sensor data as an input;

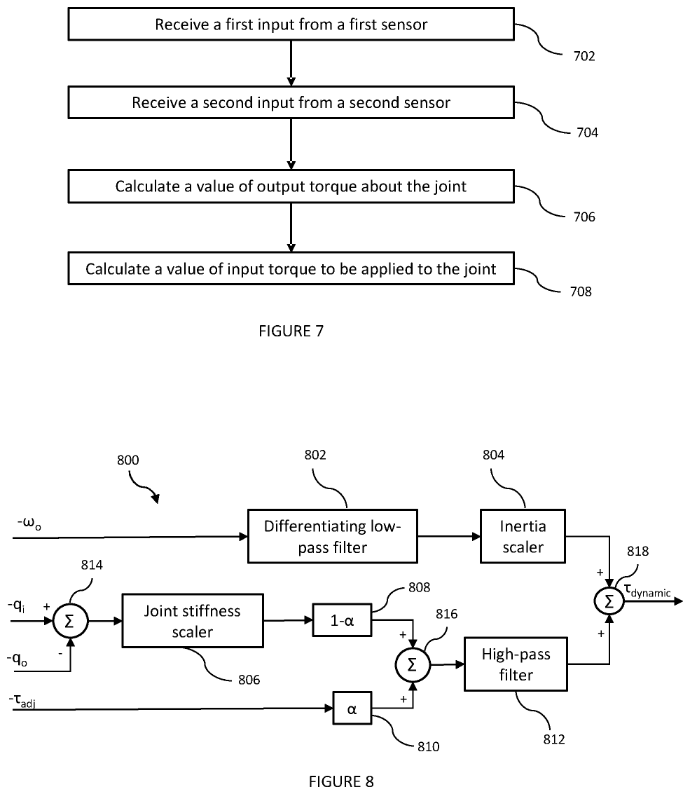

FIG. 7 illustrates a method for controlling a robotic joint using the control algorithm of FIG. 6;

FIG. 8 is a block diagram illustrating a dynamic torque observer for a robot arm for use in combination with the control algorithm of FIG. 6.

The following description describes the present techniques within the context of robotic systems. Robotic systems can include manufacturing systems, such as vehicle manufacturing systems, parts handling systems, laboratory systems, and manipulators such as for hazardous materials or surgical manipulators. The robotic system illustrated in FIG. 1 is specifically a surgical robotic system. However, the features described below are not limited to such a system but are applicable to robotic systems more generally.

FIG. 1 illustrates a surgical robot having an arm 100 which extends from a base unit 102. The arm comprises a plurality of rigid limbs 104a-e which are coupled by a plurality revolute joints 106a-e. The revolute joints 106a-e are configured to apply rotational motion. Instead of or in addition to these revolute joints, the surgical robot may comprise a one or more joints for providing linear motion. The limb that is closest to the base 102 is the most proximal limb 104a and is coupled to the base by a proximal joint 106a. The remaining limbs of the arm are each coupled in series by a joint of the plurality of joints 106b-e. A wrist 108 may comprise four individual revolute joints. The wrist 108 couples one limb (104d) to the most distal limb (104e) of the arm. The most distal limb 104e carries an attachment 110 for a surgical instrument 112. Each joint 106a-e of the arm 100 has one or more drive sources 114 which can be operated to cause rotational motion at the respective joint. Each drive source 114 is connected to its respective joint 106a-e by a drivetrain which transfers power from the drive source to the joint. In one example the drivetrain comprises one or more gears. In a more specific example, the drivetrain is a harmonic drive. A harmonic drive comprises the concentric arrangement of a wave generator, a flex spline and a circular spline, and increases the torque that can be applied to the joint by the drive source.

In one example, the drive sources 114 are motors. The drive sources 114 may alternatively be hydraulic actuators, or any other suitable means. Each joint 106a-e further comprises one or more configuration and/or force sensor 116 which provides information regarding the current configuration and/or force at that joint. Where the joint is a revolute joint, one or more of the sensors 116 may be a torque sensor. Where the joint is configured to provide linear motion, one of the one or more sensors 116 may be a strain gauge. In addition to configuration and/or force data, the one or more sensors 116 may additionally provide information regarding the temperature, current or pressure (such as hydraulic pressure). In one example, the one or more configuration sensors are position sensors. That is, the configuration sensors may measure the physical position of the joint. In such an example, one or more of the sensors 116 may be an encoder. An encoder is a position sensor which transforms a position measurement into an electronic signal. The encoder 116 may be a linear or a rotary encoder.

The drive sources 114 are arranged proximally of the joints whose motion they drive, so as to improve weight distribution. For clarity, only some of the drive sources and sensors are illustrated in FIG. 1. It can also be appreciated that the surgical robot 100 may comprise more or less limbs and joints than those illustrated in FIG. 1.

The instrument 112 of the surgical robot 100 comprises an end effector for performing an operation. The end effector may take any suitable form. For example, the end effector may be smooth jaws, serrated jaws, a gripper, a pair of shears, a needle for suturing, a camera, a laser, a knife, a stapler, a cauteriser or a suctioner. The instrument further comprises an instrument shaft and an articulation located between the instrument shaft and the end effector. The articulation comprises several joints which permit the end effector to move relative to the shaft of the instrument.

Configuration controllers for the drive sources 114 and sensors 116 are distributed within the robot arm 100. The controllers are connected via a communication bus to a control unit 118. The control unit 118 comprises a processor 120 and a memory 122. The memory 122 stores, in a non-transient way, software that is executable by the processor 120 to control the operation of the drive sources 114 to cause the arm 100 to operate. In particular, the software can control the processor 120 to cause the drive sources (for example via distributed controllers) to drive in dependence on inputs from the sensors 116 and from a surgeon command interface 124. The control unit 118 is coupled to the drive sources 114 for driving them in accordance with outputs generated by execution of the software. The control unit 118 is electrically connected to the sensors 116 for receiving sensed input from the sensors, and to the command interface 124 for receiving input from it. The electrical connections described with reference to FIG. 1 and all subsequent figures may, for example, each be electrical or optical cables, or may be provided by a wireless connection.

The command interface 124 comprises one or more input devices whereby a user can request motion of the end effector in a desired way. The input devices, or input controllers, could, for example, be manually operable mechanical input devices such as control handles or joysticks, touch-operable inputs such as touchscreens, or contactless input devices such as optical gesture sensors or voice sensors. The input devices might monitor eye movement to receive an input. The input devices could, for example, be some combination of these types of input devices. Commands input by the input devices can include movement commands, for example to move the instrument in a particular way, such as a lateral movement and/or a rotation. Such commands can include end effector commands, for example to control an end effector coupled to a distal end of the instrument 112 to operate the end effector, such as to open/close gripper jaws or to operate (turn on or off) an electrosurgical end effector.

The software stored in the memory 122 is configured to respond to those inputs and cause the joints of the arm and instrument to move accordingly, in compliance with a pre-determined control strategy. The control strategy may include safety features which moderate the motion of the arm and instrument in response to command inputs. Thus, in summary, a surgeon at the surgeon console which suitably comprises the command interface 124 can control the instrument 112 to move in such a way as to perform a desired surgical procedure. Thus, the robot arm 100 functions as a master-slave manipulator where the control unit 118 acts as a master controller. The control unit 118 and/or the command interface 124 may be remote from the arm 100.

FIG. 2 illustrates an idealised control loop 200 for controlling the configuration of a revolute joint in a robot arm as illustrated in FIG. 1. The control loop comprises a configuration controller 202, a torque controller 204, an inertia output 206 and a sensor 208.

The controller 202 may be distributed within a robot arm, such as the arm illustrated in FIG. 1, such that it is proximal to a joint that it is configured to control. The configuration controller 202 is electrically connected to the one or more sensor 208 by means of a feedback loop. The feedback loop enables the controller 202 to receive measurements indicating the configuration of the joint from the sensor. The configuration controller 202 is also connected to the control unit 118 by means of a feedforward loop and is configured to receive a reference configuration value q_{ref} from the control unit 118. In one example, the configuration controller 202 is a separate entity to the control unit 118. In another example, the configuration controller 202 and the control unit 118 are comprised within the same entity. Thus, in this example, the control unit may be directly connected to the sensor 208.

The reference configuration q_{ref} may otherwise be referred to as the desired or commanded configuration of the joint. The reference configuration q_{ref} is calculated from an end effector pose that is commanded by the surgeon at the command interface 124. The reference configuration q_{ref} may be calculated by applying one or more inverse kinematic calculations to the commanded end effector pose. In one example, the reference configuration q_{ref} is the desired physical position of the joint.

The configuration controller 202 is configured to perform a calculation using the reference position value q_{ref} received from control unit 118 and the feedback value from the sensor 208 to generate a reference torque value τ_{ref}. The reference torque value τ_{ref} indicates the demanded torque that is required to move the joint to the reference configuration q_{ref}. The reference torque value τ_{ref} is then transmitted to the torque controller 204.

The torque controller 204 is electrically connected to the configuration controller 202 and is configured to receive the reference torque value τ_{ref}. From this value of τ_{ref}, the joint torque controller is configured to calculate a value of desired current that is to be generated by the drive source. This desired current is used to generate an output torque τₒ, which is the actual torque that is experienced by the joint when it is driven by the drive source. In an idealised system, the drivetrain that transfers torque from the drive source to the joint is “stiff” and so no torque is “lost” as it is transferred from the drive source to the joint. Thus, the output torque τₒ that is experienced at the joint is equal to the reference torque τ_{ref} that is received by the configuration controller 202.

A joint that is not stiff, when it is driven, experiences an inertia which opposes the output torque τₒ provided by the drive source. The inertia may be caused by a number of factors that are either internal or external to the joint. An exemplary cause of inertia is the weight of the joint due to gravity. Another exemplary cause is the weight of an object being supported by or interfacing with the joint. Due to presence of inertia, the configuration of the joint at its output qₒ may differ from the reference configuration q_{ref}. For a joint that is stiff, the inertia will not have an effect on the output configuration of the joint q_{ref}. Thus, for a stiff joint, there will be no difference between the output configuration qₒ and the desired configuration q_{ref}.

Sensor 208 is provided to measure the output configuration qₒ of the joint. The sensor 208 may correspond to one of the sensors 116 illustrated in FIG. 1. Thus, the sensor 208 may be comprised within a joint of the robot arm 100. In one example the output configuration qₒ is the output position of the joint. The sensor 208 may therefore be a position sensor such as a linear or a rotary encoder. In this example, the sensor 208 is configured to measure the position of the joint within which it is comprised, and to transmit an electrical signal indicating this measurement to the configuration controller 202. The transmittance of this electrical signal forms a feedback loop that is used by the configuration controller 202 to calculate an updated value reference torque τ_{ref}.

Generally, feedback terms are used within control systems to indicate a deviation between the desired configuration of a joint and its actual configuration, and to compensate for disturbances that may take the joint further from steady state. Thus, the value of reference torque τ_{ref} that incorporates feedback from the sensor 208 may be continuously updated as continuous data measurements are obtained from the sensor 208. The feedback loop may further comprise one or more filters, integrators or differentiators for manipulating the raw output value that is obtained from the sensor 208 before it is provided to the configuration controller 202.

As mentioned above, the joint to be controlled by the loop illustrated in FIG. 2 has been modelled as “stiff”. For stiff joints, the input torque (i.e. the torque that is measured at, or at a position proximal to, a first end of the drive train at which the drive source is located) is proportional to the output toque (i.e. the torque that is measured at, or at a position proximal to a second end of the drive train at which the joint is located).

The output torque measured about the joint can be characterised by the following equation:

τₒ=K(qᵢ−qₒ)

Where K is the spring constant, or stiffness, of the drivetrain, qᵢ is the input configuration of the joint and qₒ is the output configuration of the joint. In one example, as illustrated in FIGS. 4 to 6 below, qᵢ is measured at the first end of the drivetrain and qₒ is measured at the second end of the drivetrain. Thus, the output torque is characterised by a relationship between the elongation (qᵢ−qₒ), and stiffness K of the drivetrain. In some examples, the relationship between elongation and stiffness K is linear. In other examples, this relationship is non-linear.

The spring constant K referenced herein may otherwise be referred to as a spring coefficient. It is noted that the spring constant (or spring coefficient) does not necessarily have a constant value for all elongation values. Indeed, in some examples, the spring coefficient is a continuous function of the elongation value, and in some other examples the spring coefficient is selected from a set of discrete values, with each discrete value being associated with a respective range of elongation values.

As described above, mechanisms for driving joints within a robotic system comprise an associated element of elasticity, which results in a non-linear stiffness characteristic. This elasticity is especially characteristic of harmonic drives. FIG. 3 is a graph illustrating an exemplary non-linear stiffness characteristic for an elastic joint in a robot arm. The graph plots elongation on the x-axis and output torque on the y-axis. The output torque may be measured by a sensor located at a second end of the drivetrain, at which the joint is located. The sensor for measuring output torque may be a gauge sensor. A gauge sensor is configured to measure changes in voltage due to mechanical strain. Elongation is calculated by subtracting the input configuration qᵢ from the output configuration qₒ of the joint. In FIGS. 4 to 6, input configuration is measured by a first sensor located at a first end of the drivetrain at which the drive source is located. Output configuration is measured by a second sensor located at a second end of the drivetrain at which the joint is located.

In FIG. 3, output torque τ₀ is measured in Nm and elongation ε is measured in degrees. In an alternative example, elongation ε may be measured in radians. In both of these examples, the first and second sensors for measuring the input and output configurations are rotary position sensors. In a further example, elongation may be a linear measurement that may be calculated using measurements obtained from linear position sensors. In this example, elongation may be measured in meters. In a further example, elongation may be dimensionless. An example of a dimensionless measurement of elongation is as a percentage.

The graph illustrated in FIG. 3 comprises five distinct regions 302-310. Regions 302 and 310 demonstrate a directly proportional relationship between drivetrain elongation and output torque τₒ. That is, as the elongation of the drivetrain increases, the output torque increases at a constant rate. This represents the idealised performance of the joint as described above with reference to FIG. 2. Region 302 illustrates the joint performance for negative values of elongation, and region 310 illustrates its performance for positive values of elongation. The gradient of the graph plotted in regions 302 and 310 represents the stiffness K of the drivetrain within the associated range of elongation values.

Region 306 represents the phenomenon of backlash, where the gradient of line representing the stiffness of the drivetrain is near-zero. As described above, backlash refers to the lost motion in a mechanism that is experienced when it changes direction and is caused by gaps or clearance between interfacing components of the drivetrain. For a harmonic drive, backlash is the result of clearance between the circular spline and the flex spline, and/or elastic deformation of the flex spline under torque. This phenomenon typically occurs within a region of elongation values. In the example illustrated in FIG. 3, this region of elongation values is between −0.1 and 0 degrees. It will be appreciated that, for different joints, the backlash region will present itself at a different range of elongation values. In this region of the graph the measured sensor torque is zero and remains at zero as the elongation increases between −0.1 and 0 degrees. Thus, during the reversal of the joint direction between −0.1 and 0 degrees of elongation no torque is transmitted through the drivetrain from the drive source to the joint.

Regions 304 and 308 are transition zones. In these zones the drivetrain is transitioning between the backlash region and its regions of intended performance. During these regions, there is a non-linear relationship between elongation and sensor torque.

The representative stiffness of the drivetrain throughout the range of elongation values recorded in FIG. 3 is represented by the gradient of a regression line 312 that passes through all regions of the graph. It is appreciated from FIG. 3 that, for an elastic joint, the relationship between elongation and output torque is not entirely linear. That is, the gradient of the stiffness characteristic in the backlash region 306 (which is zero) differs substantially from its gradient in regions 302 and 310. Thus, the straight line represented by 312 does provide an accurate representation of the stiffness characteristic of the drivetrain for all values of elongation.

The performance of joints demonstrating non-linear characteristics as illustrated in FIG. 3 cannot be accurately modelled. It is therefore important for robotic control systems to incorporate feedback loops that measure the real-world performance of the joint and use those performance measurements as feedback terms to adjust the reference signals that are used to drive the joint.

The differential equations for a single elastic joint may be expressed as follows:

M\ddot{q}ₒ+D(\dot{q}ₒ−\dot{q}ᵢ)+K(qₒ−qᵢ)+Fₒ=0

B\ddot{q}ₗ+D(\dot{q}ₗ−\dot{q}ₒ)+K(qᵢ−qₒ)+Fᵢ=τₒ

In the above equations, M, B, K, D, Fᵢ and Fₒ are control variables. M is the inertia at the output of the drivetrain, Bₒ is the inertia at its input (i.e. at the output of the drive source), K is the spring constant for the drivetrain, and D is a damping term. D, therefore, represents the energy lost due to the elasticity in the drivetrain. Fᵢ is the input torque disturbance. Input torque disturbance may be due to static friction, for example. Fₒ is the output torque disturbance. In one example, output torque disturbance is caused by the other joints in the robot arm.

In addition to the abovementioned control variables, the differential equations observe four state variables for an elastic joint: position, velocity, acceleration and torque. These variables may be defined at either the input or output of the drivetrain. In the above equations the output configuration of the joint qₒ is an output position of the joint. Thus, \dot{q}ₒ and \ddot{q}ₒ are the first and second derivatives of the output position, respectively, with respect to time. That is, \dot{q}ₒ and \ddot{q}ₒ are the velocity and acceleration, respectively, at the second end of the drivetrain. Correspondingly, qᵢ, \dot{q}ₗ and \ddot{q}ₗ are the position, velocity and acceleration at the first end of the drivetrain. The differential equations above can be parameterised by measuring the state variables described above using position and torque sensors located the first and second ends of the drivetrain. Velocity and acceleration are derivatives of position with respect to time, and so can be calculated from position measurements measured from position sensors. Torque data values are traditionally obtained from torque sensor measurements.

FIG. 4 is a block diagram illustrating a full state control scheme for controlling the configuration of a joint in a robot arm. In the specific example illustrated in FIG. 4, the configuration of the joint is its position. The control scheme may be comprised within the joint controllers described with reference to FIG. 1. The control scheme comprises a torque controller 402 and a control loop 404 that are electrically connected to each other by interconnect circuitry. The joint controller 402 comprises one or more electrical connections for connecting to the joint 406 of the robot arm. The robot arm may correspond to the arm 100 illustrated in FIG. 1, and the joint 406 may correspond one or more of joints 104a-e.

The torque controller 402 is configured to calculate a value of input torque τᵢ to be generated by the drive source and transmitted by the drivetrain to the joint 406. The control loop 404 is configured to receive a sensor data from the joint 406 and to calculate a reference torque value τ_{ref} using this sensor data as feedback.

The torque controller 402 comprises a drive source 408 and a drive source controller 410. The drive source 408 is connected to both the joint 406 of the robot arm and to the drive source controller 410. The drive source 408 is configured to supply an input torque τᵢ to the drivetrain that drives the joint 406 of the robot arm. In one example, the drive source 408 is a motor. In other examples the drive source 408 may be a hydraulic or pneumatic actuator, or any alternative power source. The drive source controller 410 is configured to control the performance of the drive source, and therefore the input torque about the joint 406.

The drive source 408 and the drive source controller 410 are electrically connected to each other by means of a feedforward loop passing from the controller to the drive source, and a feedback loop passing from the drive source to the controller. In an example where the drive source is a motor, the feed forward loop enables the controller to provide a three-phase voltage supply to the motor. In this example, the feedback loop enables the motor to supply a three-phase current feedback to the controller. The feedback loop is used to modify the voltage, and therefore the current, that is supplied to the motor. Thus, the feedback loop ensures that the intended torque is being generated by the motor.

The torque controller 402 further comprises a plurality of sensors for measuring the one or more parameters indicating the performance of the joint 406. The sensors are further configured to generate electrical signals associated with their measurements, such that these electrical signals can be transmitted to the control loop 404. In one example, the sensors are comprised within the joint 406. In another embodiment the sensors are external, but coupled, to the joint 406. The plurality of sensors may correspond to the sensors 116 illustrated in FIG. 1. In FIG. 4, the plurality of sensors comprises a first sensor 412, a second sensor 414 and a third sensor 416.

The first sensor 412 and the second sensor 114 are configured to measure the configuration about the joint 406. As mentioned above, the configuration of the joint may be the position of the joint. In this example, the sensors may be encoders. The encoders may be either linear encoders or rotary encoders, depending on the motion of the joint to which they are coupled. The first sensor 412 is configured to measure the input configuration of the joint 406. The input position is measured at the output of the drive source that is configured to drive that joint. Thus, the first sensor 412 may be located at, or at a position proximal to, a first end of the drivetrain, at which the drive source 408 is located. The second sensor 414 is configured to measure the output configuration at the joint 406. The output configuration is measured at the input to the joint. Thus, the second sensor is located at, or at a position proximal to, a second end of the drivetrain, at which the joint 406 is located. The third sensor 416 is configured to measure the output torque about the joint 406. Thus, the third sensor is also located at the second end of the drivetrain. The sensors 412, 414, 416 may be configured to continuously sense data indicating the performance of the joint. This continuous sensing of data may be performed at regular intervals. That is, the sensing of data from the joint may be performed by the sensors at a predefined sampling rate. This sampling rate may be configurable, such as at the command interface 124 illustrated in FIG. 1.

In addition to the one or more sensors, the torque controller 402 comprises one or more sensor filters for filtering electrical signals received from a respective sensor. Filters are provided to combine information from multiple data values to efficiently monitor the performance of the joint. In one example, the filters are temporal filters, which are used to incrementally update their information for each new data value. The first filter 418 is configured to filter electrical signals received from the first sensor 412. Where the first sensor 412 is an input position sensor, the first filter 418 is configured to filter input position values.

Correspondingly, a second filter 422 is electrically connected to the second sensor 414 and is configured to filter electrical signals received from that sensor. Thus, where the second sensor 414 is an output position sensor, the second filter 422 is configured to filter output position values. A third filter 426 is electrically connected to the third sensor 416. The third filter 426 is therefore configured to filter output torque values.

The torque controller 402 further comprises fourth and fifth filters 420, 424 for deriving filtered derivative values from the first and second sensors 412, 414 respectively. As mentioned above, the sensing of data by the sensors 412, 414, 416 may be performed at a predefined sampling rate. The fourth and fifth filters 420, 422 are configured to maintain a record of the sampling rate at which sensor data is measured. Velocity is the first derivative of position with respect to time. Thus, where the first and second sensors 412, 414 are input and output position sensors, the fourth and fifth filters 420, 422 are configured to calculate input and output velocity values for the joint, respectively, using the predefined sampling rate and the continuously sampled data values.

As well as being electrically connected to the one or more sensors, the one or more sensor filters are electrically connected to incoming interconnect circuitry 428. The incoming interconnect circuitry 428 is further connected to the control loop 404. The interconnect circuitry 428 is therefore configured to transmit sensor data from the torque controller 402 to the control loop 404. Thus, the sensor data may be provided as feedback to the control loop 404. In one example, the interconnect circuitry is a communication bus, such as an Ethercat Bus.

The control loop 404 is configured to calculate a reference torque τ_{ref} to be provided to the drive source 408. The reference torque τ_{ref} can be calculated using a reference configuration q_{ref} and a plurality of feedback loops derived from measured sensor data. The control loop 404 comprises a sixth filter 430, a configuration controller 432, a dynamic torque observer 434 and a gravity compensator 436.

The sixth filter 430 is connected to the configuration controller 432 and is configured to receive one or more incoming reference configuration value q_{ref} (such as from control unit 118). The sixth filter 430 is further configured to filter these values to provide as an input to configuration controller 432. The filter 430 is also configured to calculate a value for the first derivative of this reference configuration q_{ref}. Where the reference configuration is a reference position for the joint 406, the first derivative of this reference configuration (with respect to time) is reference velocity. The sixth filter 430 may correspond to the filters 418, 420, 422, 424, 426 of the torque controller 402. That is, the position filter may be configured to combine information from multiple data values to efficiently monitor the input position values, and to calculate a reference velocity value using a stored predefined sampling rate and the continuously sampled reference position values.

The control loop further comprises a gravity compensator 436, which is electrically connected to both the output of the sixth filter 430 and the incoming interconnect circuitry 428. The gravity compensator 436 is configured to receive reference configuration data from the sixth filter 430 and output torque values from the third sensor 416 by means of the interconnect circuitry 428. The gravity compensator 436 is further configured to calculate an expected torque value using the data obtained from sixth filter 430. A value gravity-compensated measured torque can then be calculated by the gravity compensator 436 by subtracting expected torque from output torque τₒ. This gravity-compensated measured torque is provided to dynamic torque observer 434.

In addition to the gravity-compensated measured torque, the dynamic torque observer 434 is configured to receive values of input configuration, output configuration and the first derivative of output configuration from the interconnect circuitry 428. The dynamic torque observer 434 is further configured to calculate a dynamic torque value τ_{dynamic} from its received inputs. The dynamic toque value τ_{dynamic} is transmitted by the dynamic torque observer 434 to the configuration controller 432. The configuration controller 432 is also electrically connected to the sixth filter 430 and interconnect circuitry 428. The configuration controller 432 is therefore configured to receive, in addition to τ_{dynamic}, the input and output configurations, and the first derivatives of these configurations, from the incoming interconnect circuitry 428. The position controller is further configured to receive the reference configuration and first derivative of this value from the sixth filter 430. From these received values, the configuration controller 432 calculates a value for reference torque τ_{ref} to be provided to the torque controller 402. The reference torque τ_{ref} is transmitted from the command loop 404 to the torque controller 402 by outgoing interconnect circuitry 434. As with incoming interconnect circuitry 428, outgoing interconnect circuitry 434 may be a communication bus, such as an Ethercat bus.

FIG. 5 is a block diagram illustrating a control algorithm 500 for controlling the configuration of a robotic joint, and that uses torque sensor data as a feedback term to calculate a value of input torque τᵢ. The position control algorithm may be comprised within the configuration controller 432 illustrated in FIG. 4, for example. The position control algorithm uses a reference configuration q_{ref} (received from control unit 118, for example) and a plurality of feedback terms to calculate an input torque value τᵢ to be provided to a drive source. The reference configuration q_{ref} is initially multiplied by a first transfer function 502 and is provided to first summing point 504. The first transfer function 502 comprises a first gain that is provided to manipulate the raw input value of q_{ref}.

The control algorithm 500 further comprises a second transfer function 506 which comprises a gain G(s) that is associated with the drive source. In an example where the drive source is a motor, the gain G(s) is the motor gain. The value of motor gain G(s) is dependent on the physics of the joint. A value of physical torque τₚ and input torque τᵢ are provided as inputs to the second transfer function 506. The physical torque τₚ that is applied to the second transfer function 506 represents the real-life disturbance to the joint system. This torque does not exist electronically. The outputs of the second transfer function 506 are output position qₒ, input position qᵢ and output torque τₒ. These outputs are measured parameters that are obtained from sensors located at the joint and its respective drivetrain. The sensors may, for example, correspond to sensors 412, 414, 416 illustrated in FIG. 4.

The three outputs qₒ, qᵢ, τₒ from transfer function 506 that are measured at the joint are provided as feedback terms that are used to calculate the input torque τᵢ. Each measured parameter is provided with an associated feedback loop. Each feedback loop comprises a transfer function that corresponds to the output value with which it is associated. For example, the feedback loop for measured output configuration qₒ comprises a third transfer function 508. In the example illustrated in FIG. 5 the third transfer function comprises one associated gain Kₒ. In an alternative example, the third transfer function 508 comprises more than one gain. The third transfer function 508 is configured to calculate manipulated values of both the output configuration and the first derivative of output configuration \dot{q}ₒ to provide to summing point 510. In the example in which the transfer function comprises more than one gain, a gain may be provided for each derivative of the output configuration provided as an input to the transfer function 508. So, where the third transfer function is configured to calculate manipulated values of both the output configuration and the first derivative of the output configuration, the third transfer function 508 may comprise two gains. In a further alternative example, the third transfer function 508 may comprise a matrix, where each element of the matrix is a function relating a particular input variable to an output variable.

The feedback loop provided for measured input configuration qᵢ comprises a fourth transfer function 512. Similarly to the third transfer function 508, in the example illustrated in FIG. 5 the fourth transfer function comprises one associated gain Kᵢ. In an alternative example, the fourth transfer function 512 comprises more than one gain. Specifically, where the fourth transfer function is configured to calculate manipulated values of both the input configuration and the first derivative of the input configuration, the fourth transfer function 512 may comprise two gains. In a further alternative example, the fourth transfer function 512 may comprise a matrix, where each element of the matrix is a transfer function relating a particular input variable to an output variable.

The fourth transfer function 512 is configured to calculate manipulated values of both the input configuration and the first derivative of input configuration \dot{q}ₗ to provide to summing point 514. The feedback loop provided for output torque τₒ comprises a fifth transfer function 516 with an associated gain K_{τ}. A manipulated value of output torque τₒ is thus provided to summing point 514. The gains Kₒ, Kᵢ, K_{τ} may be predefined continuous or constant values that may be obtained from predetermined theoretical or experimental data. The gains may be stored in a non-transient form within the control system for the robot arm. The gains may be applied to the physical measurement data and are used to affect the performance of the control algorithm.

The feedback loops are summed together at summing points 510, 514, and are used to calculate the input torque τᵢ at summing point 504. That is, summing point 504 receives manipulated reference configuration q_{ref} and the manipulated sensor data feedback as inputs, and outputs input torque τᵢ. Knowing the configuration of the joint at the input and at the output of the drivetrain allows the control system to determine the elongation drivetrain. Thus, the joint can be controlled in dependence on that elongation.

Using the control algorithm illustrated in FIG. 5, the value of input torque τᵢ calculated by the controller is represented by the following equation:

τᵢ=kₚₒ(q_{ref}−qₒ)+kₚᵢ(q_{ref}−qᵢ)+k_{do}(\dot{q}_{ref}−\dot{q}ₒ)+k_{di}(\dot{d}_{ref}−\dot{q}ᵢ)−Kₜτₒ

In the above equation, q_{ref} is a reference configuration that is computed for each joint of the arm. The configuration may be angular or linear, depending on the motion of each joint. kₚₒ, kₚᵢ, k_{do} and k_{di} are gains for the first and second inputs. kₚₒ and kₚᵢ are proportional gains for the input and output configurations respectively. k_{do} and k_{di} are derivative gains for the input and output configurations respectively. The first derivative of reference and measured configurations \dot{q}_{ref}, \dot{q}ᵢ and \dot{q}ₒ are synonymous with the respective velocities ω_{ref}, ωᵢ and ωₒ

The control system illustrated in FIG. 5 requires five measured feedback terms (qᵢ, qₒ, \dot{q}ₗ, \dot{q}ₒ, τₒ) to calculate input torque τᵢ. However, measured output torque τₒ cannot always be used to provide an accurate representation of the performance of a joint. This is of particular relevance to joints with drivetrains which experience backlash, such as harmonic drives. Generally, the elongation of the drivetrain varies linearly with variation in the torque applied to that joint. As described above, in the backlash region of a non-linear joint the measured sensor torque is near-zero as the joint elongation varies. Thus, during the backlash region torque sensors cannot be used to provide values of measured torque. There is a need for a more accurate method of determining output torque to be provided to the controller during this region.

As introduced above, the output torque for a robotic joint can be characterised by the following equation:

τₒ=K(qᵢ−qₒ)

Where K is the spring constant of the joint, qᵢ is the input configuration about the joint and qₒ is the output configuration about the joint. As previously mentioned, qᵢ−qₒ represents the elongation of the drivetrain. qᵢ and qₒ can both be measured using sensors coupled to the joint and its associated drivetrain. Thus, the output torque for the joint can be calculated using measured sensor data for the input and output joint configurations. This calculation of output torque can be used to replace a measured value of output torque obtained from a torque sensor.

FIG. 6 is a block diagram illustrating a control algorithm for controlling the configuration of a joint without using torque sensor data as a feedback term. Instead, the above equation linking output torque to the input and output joint configurations is used to calculate a value of output torque to provide feedback to the control system. The controller for performing the configuration control algorithm may be implemented within a control scheme that broadly corresponds to the scheme illustrated in FIG. 4 but excluding torque sensor data obtained from the third sensor 416. The control algorithm is configured to be implemented by a controller similar to the controller 432 illustrated in FIG. 4.

The control algorithm comprises three transfer functions: first transfer function 602, second transfer function 606 and third transfer function 610. The algorithm further comprises a first summing point 604 and a second summing point 608. As with the algorithm illustrated in FIG. 5, the control algorithm receives a reference configuration q_{ref}, and uses this reference configuration and a plurality of feedback terms to calculate an input torque value τᵢ.

The reference configuration q_{ref} is received from a control unit, such as the unit 118 in FIG. 1. The reference configuration q_{ref} is provided as an input to a first transfer function 602. The first transfer function 602 is described in more detail below. The second transfer function 606 comprises a gain G(s) that is associated with the drive source. A value of physical torque τₚ and input torque τᵢ are provided as inputs to the second transfer function 606. The outputs of the second transfer function 606 are output configuration qₒ and input configuration qᵢ. These outputs are measured parameters that are obtained from sensors located at the joint and its respective drivetrain.

In one example, as illustrated in FIG. 6, the third transfer function 610 comprises two values, kₜ and kφ. kφ is the spring constant of the drivetrain and may be synonymous with K as referenced above. kφ is described in greater detail below. kₜ is a gain provided to manipulate the raw values of qₒ and qᵢ. In an alternative example, the third transfer function 610 is a matrix. In this example, each element of the matrix is a function relating a particular input variable to an output variable.

A method for controlling a robotic joint using a controller implementing the control algorithm of FIG. 6 is illustrated in FIG. 7. At step 702, the controller receives a first input from a first sensor. The first input indicates the input configuration of the joint qᵢ. The first sensor may correspond to first sensor 412 in FIG. 4. As mentioned above with reference to FIG. 4, the first sensor 412 may be located at a first end of the drivetrain, at which the drive source 408 is located.

At step 704, the controller receives a second input from a second sensor. The second input indicates the output configuration of the joint qₒ. The output configuration is received from a second sensor, which may correspond to second sensor 414 in FIG. 4. In one example, the second sensor 414 may be located at the second end of the drivetrain at which the joint is located.

At step 706, the controller is configured to calculate a value of output torque τₒ about the joint using the first and second inputs. At step 708, the controller calculates a value of input torque to be applied to the joint using the calculated value of output torque. In one example, calculating the input torque comprises subtracting a torque term which compensates for the action of gravity from the calculated value of output torque τₒ. The use of a torque term which compensates for the action of gravity to calculate input torque prevents the drifting of the joint due to gravity during its operation.

In FIG. 6, both the first input and the second input are received as inputs to the third transfer function 610. In one example, the first input and the second input are each scaled by a gain before being provided to the third transfer function 610. In this example the gain by which the first input is scaled may be different from the gain by which the second input is scaled. The third transfer function 610 is further configured to calculate a value of output torque τₒ using the received values of output configuration qₒ and input configuration qᵢ for the joint. This output torque τₒ corresponds to an idealised value of torque which may be different from the value of torque that would be measured by a torque sensor. That is, when the joint is experiencing backlash, the measured value of output torque for a joint τₒ would be zero. However, the value of output torque τₒ that is calculated using input and output configuration values will have a non-zero value under these conditions. The calculated value of output torque τₒ that is provided as an output from the third transfer function 610 is provided as an input to summing point 604. The output of first transfer function 602 is also provided to summing point 604.

The summing point 604 is to calculate a value of input torque τᵢ using the calculated value of output torque, together with the output of the first transfer function 602. This calculation is performed at step 708 in FIG. 7. In the example illustrated in FIG. 6 the first transfer function 602 comprises one associated gain K_{PD} that corresponds to gains K ₒ and Kᵢ of transfer functions 508 and 512 illustrated in FIG. 5. In an alternative example, the first transfer function 602 comprises a matrix, where each element of the matrix is a transfer function relating a particular input variable to an output variable. Thus, transfer function 602 is configured to receive measured values of qₒ and qᵢ as inputs, and to provide manipulated versions of these configurations and their derivatives to summing point 604. In another example, the first transfer function 602 comprises more than one gain. Where the transfer function 602 is configured to receive measured values of qₒ and qᵢ as inputs, it may comprise two gains. In this example, a first gain is provided to manipulate output configuration qₒ, and a second gain is provided to manipulate input configuration qᵢ.

The control algorithm described in FIG. 6 may receive a continuous stream of sensor data from the indicating the input and output configurations of the joint. Thus, the control algorithm may be configured to repeatedly calculate the value of input torque τᵢ. The continuous calculation of input torque τᵢ ensures that the control system is able to instantaneously compensate for disturbances to the joint performance as they occur. In one example, this compensation may occur in substantially real time.

Using the control algorithm illustrated in FIG. 6, the value of input torque τᵢ calculated by the controller is represented by the following equation:

τᵢ=kₚₒ(q_{ref}−qₒ)+kₚᵢ(q_{ref}−qᵢ)+k_{do}(\dot{q}_{ref}−\dot{q}ₒ)+k_{di}(\dot{q}_{ref}−\dot{q}ᵢ)−kₜkφ(qᵢ−qₒ)

In the above equation, q_{ref} is a reference configuration that is computed for each joint of the arm. The configuration may be angular or linear, depending on the motion of each joint. kₚₒ, kₚᵢ, k_{do} and k_{di} are gains for the first and second inputs. kₚₒ and kₚᵢ are proportional gains for the input and output configurations respectively. k_{do} and k_{di} are derivative gains for the input and output configurations respectively. The first derivative of reference and measured configurations \dot{q}_{ref}, \dot{q}ᵢ and \dot{q}ₒ are synonymous with the respective velocities ω_{ref}, ωᵢ and ωₒ.

The first term of the above equation, q_{ref}−qₒ represents comparison of the reference configuration to the measured output configuration of the joint. The second term, q_{ref}−qᵢ is calculated by comparison of the reference configuration to the measured input configuration of the joint. The third term \dot{q}_{ref}−\dot{q}ₒ is calculated by comparison of the first derivative of the reference configuration to the first derivative of the measured output configuration of the joint. The fourth term, \dot{q}_{ref}−\dot{q}ᵢ calculated by comparison of the first derivative of the reference configuration to the first derivative of the measured input configuration of the joint. Thus, the first four terms of the equation above are derived by comparing the desired configuration of the joint with the input and output configurations obtained from measured sensor data. That is, in dependence on the difference between the joint's desired configuration and its measured configuration, an input torque can be calculated which will tend to drive the joint towards the desired configuration. The first four terms of the equation above, together with their associated gains, are provided as outputs from the first transfer function 602.

In one example, the configuration of the joint to be controlled by the control algorithm 600 is the position of the joint. In this example, the reference configuration is the reference, or desired, physical position of the joint. The input and output configurations are the input and output positions of the joint respectively.

The spring constant kφ that is comprised within the third transfer function 610 may be synonymous with K, as referenced above. The spring constant kφ is a theoretically calculated value that is a function of drivetrain elongation qₒ−qᵢ. That is, kφ is a predefined value or range of values obtained from historic theoretical or experimental data and stored in a non-transient form within the control system for the robot arm. With reference to FIG. 3, kφ can be characterised by the gradient of a graph plotting drivetrain elongation against output torque.

In one example, the spring constant kφ is related to drivetrain elongation by a continuous function. That is, kφ may vary continuously as drivetrain elongation varies. In another example, the spring constant kφ may have a constant value. That is, kφ have the same value as drivetrain elongation varies. Alternatively, the spring constant may be selected from a discrete range of values. In this example, each discrete value of kφ may be associated with a range of elongation values, and each range of elongation values may be defined by one or more predetermined threshold values. An example where the spring constant is selected from a range of three distinct values is demonstrated below:

e<−0.1 degrees→kφ=K1

−0.1<e<0 degrees→kφ=K2

0 degrees<e→kφ=K3

In the above example, a first spring constant value K1 is used when the measured elongation is less than −0.1 degrees. That is, the first stiffness value K1 is associated with a range of elongation values that is characterised by an upper threshold value of −0.1 degrees. This range of elongation values corresponds to region 302 of the graph illustrated in FIG. 3. A second stiffness value K2 is used when the drivetrain elongation is between −0.1 degrees and 0 degrees. That is, the second stiffness value K2 is associated with a range of elongation values that is characterised by a lower threshold value of −0.1 degrees and an upper threshold value 0 degrees. This range of elongation values corresponds to region 306 of the graph illustrated in FIG. 3. That is, this range of elongation values corresponds to the backlash region as illustrated in FIG. 3. A third stiffness value K3 is used when the measured elongation is greater than 0 degrees. That is, the third stiffness value K3 is associated with a range of elongation values that is characterised by a lower threshold value of 0 degrees. This range of elongation values corresponds to region 310 of the graph illustrated in FIG. 3.

In a further example, the spring constant may be selected from a range of five distinct values. These five distinct values include the first, second and third stiffness values K1, K2 and K3 indicated above. In addition to this a fourth stiffness value K4 may be selected when the measured elongation is equal or close to −0.1 degrees. This corresponds to the transition zone demonstrated by region 304 of the graph illustrated in FIG. 3. A fifth stiffness value K5 may be used when the measured elongation is equal or close to 0 degrees. This corresponds to the transition zone demonstrated by region 308 of the graph illustrated in FIG. 3.

As described above with reference to FIG. 3, it would be appreciated that the ranges of elongation values provided above are exemplary and that alternative ranges of such values may be associated with stiffness values K1-K5.

A robotic control system may comprise both a controller as illustrated in FIG. 5 and one as illustrated in FIG. 6. That is, the control system may comprise one controller that uses measured output torque as a feedback term, and another controller that substitutes this measured value with a calculated value of output. These two controllers can be used in combination to control joints with non-linear stiffness characteristics, as different torque feedback terms provide more accurate inputs at different elongation values. Thus, for some values of elongation it may be useful to observe measured torque, and for others (e.g. within the backlash region) it may be useful it may be useful to observe calculated torque.

The controllers illustrated in FIGS. 5 and 6 may be implemented at a dynamic torque observer 800 as illustrated in FIG. 8. As described with reference to FIG. 4, the aim of the dynamic torque observer is to calculate a value of joint torque that accounts for movement and/or acceleration of the joint, whilst ignoring external forces.

The dynamic torque observer 800 comprises a differentiating low-pass filter 802, an inertia scaler 804, a joint stiffness scaler 806, a first weighting unit 808, a second weighting unit 810 and a high-pass filter 812. If further comprises summing points 814, 816 and 818.

The differentiating low-pass filter 802 is configured to receive an output velocity value \dot{q}ₒ (or ωₒ) as an input. This velocity value is obtained from differentiation of measured output configuration qₒ that is performed by the control algorithm 600. Output velocity ωₒ is used to calculate an inertial term which indicates the force required to accelerate the joint. This value is useful at low frequencies but produces excessive noise at high frequencies. Thus, low-pass filter 802 is configured to receive incoming output velocity values and to filter out the high frequency values from those received values. The values that are output from the low-pass filter 802 are provided as an input to an inertia scaler 804. The inertia scaler 804 is configured to apply a fixed inertia to the filtered output velocity values and to calculate a value of inertial torque.

The dynamic torque observer is further configured to calculate an updated value of output torque τₒ using inputs from both a controller as illustrated in FIG. 5 and a controller as illustrated in FIG. 6. Summing point 814 and joint stiffness scaler 806 are used provide a calculated value of output torque τₒ, as described with reference to FIG. 6 above the component of that torque that is due to gravity subtracted. The component of the output torque that is due to gravity may be a theoretical value. This torque may be calculated using the geometry, mass and configuration of the joint and the arm within which it is comprised. An initial value of τ_{adj} is weighted by second weighting unit 810, and the resultant manipulated value is provided to summing point 816.

The weighting units 808, 810 are provided to modify the proportion of each feedback term that is provided to summing point 816. The first weighting unit 808 comprises a first value 1−α. The second weighting unit comprises a second value α. α is a predetermined parameter, where 1≥α≥0. In one example, α is a function of drivetrain elongation. That is, α may be related to drivetrain elongation by a continuous function, or may have a constant value, or may be selected from a discrete range of values, as described above with respect to kφ. The manipulated values of calculated/measured output torque that are output from weighting units 808, 810 are provided to summing point 816.

Output torque is assumed to comprise two components: dynamic torque (which is caused by acceleration, actuator torque and friction) and externally applied torque. In order to observe dynamic torque, the externally applied torque component of output torque must be removed. This is achieved by the addition of high-pass filter 812. High-pass filter 812 receives the new value of output torque calculated from the output of summing point 816 and filters this signal to eliminate the external torque component. The manipulated output of high-pass filter 812 can then be provided to summing point 818. The summing point 818 is further configured to receive inertial torque values from the inertia scaler 804 and provide a modified value of dynamic torque τ_{dynamic} as an output.

In the examples described above, the input configuration qᵢ that is used to calculated output torque is a measured configuration obtained from a sensor located at, or at a position proximal to, a first end of the drivetrain. In another example, input configuration may be calculated using a value of reference joint configuration that has been requested at a time prior to the time at which output joint configuration is measured. Ideally, for a “stiff” joint, a prior reference configuration will be the same as the input configuration of the joint. Thus, in this example, a sensor to measure input joint configuration is not required, increasing joint compactness.

FIGS. 2 to 6, as described above, refer to the forces generated by joints as being torques. Thus, FIGS. 2 to 6 refer to the controllers for controlling the configuration of a rotary joint. In an alternative example, the joint to be controlled may be configured to provide linear motion. In this example, corresponding forces can be measured and calculated using strain gauges or other force sensors.

The control algorithm illustrated in FIG. 6 can be used to control the configuration of a joint in a robot arm that responds to externally applied forces and forces due to gravity without the use of torque sensor data as a feedback term. Thus, a control system using this algorithm does not require a torque sensor (such as the third sensor 416 in FIG. 4) to measure the output torque about for the joint. For robotic control systems comprising only the configuration controller illustrated in FIG. 6 (as opposed to an additional controller as illustrated in FIG. 5), the elimination of a sensor for measuring output torque would result in a more compact joint design, which in turn would reduce the number of assembly steps and the computational cost of the joint. The signal disturbances produced by noise within the system can also be reduced as a result of the reduction in sensor inputs.

In addition to the above, the control algorithm illustrated in FIG. 6 may also present a solution for minimising the instability in the joint control system when the drivetrain for driving the joint provides a low stiffness. That is, by minimising the number of feedback terms, the number of gains associated with these terms can be minimised. The adjustments that are required to maximise the stability of these gains is therefore reduced. The control system can therefore be used to provide a good estimate of the desired torque that will need to be delivered by a drive source to obtain a given joint response.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A controller for controlling the configuration of a joint in a surgical robot, the joint being driven by a drivetrain which transfers power from a drive source to the joint, wherein the controller is configured to:
receive a first input indicating a configuration of the drive source;
receive a second input from a first sensor, the second input indicating a measured configuration of the joint in the surgical robot;
calculate a value of output torque about the joint using the first input and the second input; and
calculate, using the value of output torque, a value of input torque to be applied to the joint in the surgical robot by the drive source.

2. The controller as claimed in claim 1, wherein the first input is received from a second sensor and indicates a measured configuration of the drive source.

3. The controller as claimed in claim 2, wherein the measured configuration of the drive source is the measured physical position of the drive source, and the measured configuration of the joint is the measured physical position of the joint.

4. The controller as claimed in claim 1, wherein the controller is further configured to receive a third input indicating a desired configuration for the joint, wherein the desired configuration is the desired physical position of the joint.

5. (canceled)

6. The controller as claimed in claim 4, wherein the value of input torque is calculated using a comparison between the first input and the third input.

7. The controller as claimed in claim 4, wherein the value of input torque is calculated using a comparison between the second input and the third input.

8. The controller as claimed in claim 4, wherein the value of input torque is calculated using a comparison between a first derivative of the first input and a first derivative of the third input.

9. The controller as claimed in claim 4, wherein the value of input torque is calculated using a comparison between a first derivative of the second input and a first derivative of third input.

10. The controller as claimed in claim 1, wherein output torque is characterised by a relationship between an elongation value of the drive train and a stiffness value of the drivetrain.

11. The controller as claimed in claim 1, wherein the value of output torque is represented by the equation τₒ=kφ(qᵢ−qₒ), where qᵢ is the first input, qₒ is the second input and kφ is a spring coefficient of the drivetrain, and wherein kφ is related to (qᵢ−qₒ) by a continuous function.

12. (canceled)

13. The controller as claimed in claim 1, wherein the value of output torque is represented by the equation τₒ=kφ(qᵢ−qₒ), where qᵢ is the first input, qₒ is the second input, and kφ is a spring coefficient of the drivetrain, and wherein kφ is selected from a discrete range of values, each value of kφ being associated with a range of elongation values that are defined by one or more predetermined threshold values.

14. The controller as claimed in claim 13, wherein the spring coefficient is selected from three distinct values in dependence on a measured elongation of the drivetrain, wherein:
a first value is selected for the spring coefficient if the value of measured elongation is below a first predetermined threshold;
a second value is selected for the spring coefficient if the value of measured elongation is above the first predetermined threshold and below a second predetermined threshold; and
a third value is selected for the spring coefficient if the value of measured elongation is above the second predetermined threshold.

15. The controller as claimed in claim 14, wherein the range of elongation values that is above the first predetermined threshold and below a second predetermined threshold corresponds to a backlash region of the joint.

16. The controller as claimed in claim 1, wherein the value of input torque is represented by the following equation:
τᵢ=kₚₒ(qᵣ−qₒ)+kₚᵢ(qᵣ−qᵢ)+k_{do}(\dot{q}ᵣ−\dot{q}ₒ)+k_{di}(\dot{q}ᵣ−\dot{q}ᵢ)−kₜkφ(qᵢ−qₒ)
Wherein: qᵢ is the first input, qₒ is the second input, qᵣ is the third input and kₚₒ, kₚᵢ, k_{do}, k_{di} and kₜ are gains associated with the first, second and third inputs.

17. The controller as claimed in claim 1, the controller being configured to repeatedly calculate the value of output torque.

18. The controller as claimed in claim 1, wherein the controller is implemented within a dynamic torque observer, the dynamic torque observer being configured to calculate a value of dynamic torque by applying a weighting to the value of output torque calculated by the controller.

19. The controller as claimed in claim 1, wherein calculating the value of input torque comprises subtracting a torque term which compensates for the action of gravity from the value of output torque.

20. (canceled)

21. The controller as claimed in claim 1, wherein the first sensor is located at, or at a position proximal to, a second end of the drive train at which the joint is located, and the second sensor is located at, or at a position proximal to, a first end of the drive train at which the drive source is located.

22. (canceled)

23. The controller as claimed in claim 1, wherein the drivetrain is a harmonic drive comprising one or more gears.

24. (canceled)

25. A method for controlling the configuration of a joint in a surgical robot, the joint being driven by a drivetrain which transfers power from a drive source to the joint, the method comprising:
receiving a first input indicating a configuration of the drive source;
receiving a second input from a first sensor, the second input indicating a measured configuration of the joint in the surgical robot;
calculating a value of output torque for the joint using the first input and the second input; and
calculating, using the value of output torque, a value of input torque to be applied to the joint in the surgical robot by the drive source.
